(19) United States
(12) Patent Application Publication
(10) Pub. No.: US 2007/0142648 A1
(43) Pub. Date: Jun. 21, 2007

(54) Hydrogenation method for producing optically active alcohols or carboxylic acids

(75) Inventors: Heiko Urtel, Frankenthal (DE); Markus Rosch, Dienheim (DE); Andrea Haunert, Mannheim (DE)

Correspondence Address: CONNOLLY BOVE LODGE & HUTZ LLP, P.O. BOX 2207, WILMINGTON, DE, 19899-2207, US

(73) Assignee: BASF Aktiengesellschaft, Ludwigshafen (DE)

(21) Appl. No.: 10/588,948

(22) PCT Filed: Feb. 8, 2005
(86) PCT No.: PCT/EP05/01234

§ 371 Date: Aug. 10, 2006

(30) Foreign Application Priority Data
Feb. 13, 2004 (DE) 10 2004 007 498.4

(51) Int. Cl. C07D 305/12; C07D 307/02; C07D 207/04; C07C 29/14

(52) U.S. Cl. 548/571; 549/326; 549/508; 564/503; 568/881

(57) ABSTRACT

The present invention relates to a process for preparing optically active hydroxy-, alkoxy-, amino-, alkyl-, aryl- or chlorine-substituted alcohols or hydroxy carboxylic acids having from 3 to 25 carbon atoms or their acid derivatives or cyclization products by hydrogenating the correspondingly substituted optically active mono- or dicarboxylic acids or their acid derivatives in the presence of a catalyst whose active component comprises a noble metal selected from the group of the metals Pt, Pd, Rh, Ir, Ag, Au and at least one further element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb and Lu.

The present invention relates to a process for preparing optically active hydroxy-, alkoxy-, amino-, alkyl-, aryl- or chlorine-substituted alcohols or hydroxy carboxylic acids having from 3 to 25 carbon atoms or their acid derivatives or cyclization products by hydrogenating the correspondingly substituted optically active mono- or dicarboxylic acids or their acid derivatives.

The target compounds mentioned constitute valuable intermediates for the pharmaceuticals and cosmetics industry for the preparation of medicaments, fragrances and other organic fine chemicals which are difficult to obtain inexpensively.

EP-A 0696575 describes a process for preparing optically active amino alcohols by hydrogenating the corresponding amino acids in the presence of Ru catalysts reduced with hydrogen at temperatures of from 50 to 150° C. and pressures of from 5 to 300 bar.

EP-A 0717023 relates to a process for preparing optically active alcohols by reducing the corresponding optically active carboxylic acids in the presence of Ru catalysts reduced with hydrogen at temperatures of <160° C. and pressures of <250 bar.

WO 99/38838 describes a process for preparing optically active amino alcohols by catalytically hydrogenating the corresponding amino acids with bi- or trimetallic unsupported or supported Ru catalysts with addition of acid.

WO 99/38613 the preparation of unsupported hydrogenation catalysts which comprise ruthenium and at least one further metal having an atomic number of from 23 to 82. Using these catalysts, it is possible to hydrogenate carboxylic acids and their derivatives under mild conditions. In the case of enantiomerically pure substrates, the achievable enantiomeric success is a maximum of 98.8% at yields below 80%.

WO 99/38824 describes a process for preparing optically active alcohols by reducing optically active carboxylic acids in the presence of Ru catalysts which have been reduced with hydrogen and comprise at least one further metal having an atomic number in the range from 23 to 82.

EP-A 1051388 describes unsupported Ru/Re suspension catalysts by which chiral α-amino acids or α-hydroxy acids can be reduced at from 60 to 100° C. and 200 bar of hydrogen pressure to the corresponding chiral alcohols.

U.S. Pat. No. 4,659,686 discloses that, using alkali metal- or alkaline-earth metal-doped catalysts which comprise a Pt group metal and Re on carbon in the hydrogenation of malic acid, the reaction products formed are tetrahydrofuran (THF) and/or butanediol (BDO). Butanetriol is not found using these catalysts.

EP-A 147 219 describes Pd—Re catalysts and their use in a process for preparing THF and BDO. Example 39 shows that the hydrogenation of malic acid at 200° C. and 170 bar leads in yields of 66% to THF and of 21% to BDO. Butanetriol is not found.

Adv. Synth. Catal. 2001, 343, No. 8 describes the use of the Nishimura catalyst (Rh—Pt oxide) for the racemization-free hydrogenation of α-amino acid esters and α-hydroxy carboxylic esters. However, large amounts (10% by weight) of the expensive catalyst system are required there. Moreover, the free carboxylic acids initially have to be converted to the corresponding esters in a further synthetic step.

A problem in the use of Ru catalysts in the hydrogenation of carboxylic-acids is that they have a high tendency to decarbonylate the reactants used or the products obtained to release carbon monoxide. In addition to the associated high pressure rise, the reduction of the carbon monoxide released to methane constitutes a great safety risk.

It is an object of the present invention to provide a process for hydrogenating optically active carboxylic acids or their acid derivatives to the corresponding optically active alcohols, in which the undesired decarbonylation of the reactants used or the products formed is very substantially prevented.

According to the invention, this object is achieved by providing a process for preparing optically active hydroxy-, alkoxy-, amino-, alkyl-, aryl- or chlorine-substituted alcohols or hydroxy carboxylic acids having from 3 to 25 carbon atoms or their acid derivatives or cyclization products by hydrogenating the correspondingly substituted optically active mono- or dicarboxylic acids or their acid derivatives in the presence of a catalyst whose active component comprises a noble metal selected from the group of the metals Pt, Pd, Rh, Ir, Ag, Au and at least one further element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu.

The process according to the invention is suitable for hydrogenating optically active mono- or dicarboxylic acids having from 3 to 25, preferably having from 3 to 12, carbon atoms, which may be straight-chain, branched or cyclic and have at least one, typically from 1 to 4, substituents each bonded to an asymmetrically substituted carbon atom. The process is equally suitable for hydrogenating acid derivatives of the substituted carboxylic acids mentioned. Here, as within the entire context of the present invention, the term acid derivative means that the acid function is present in the form of an ester, a partial ester, an anhydride or an amide, preferably in the form of an ester or partial ester.

In the context of the present invention, optically active compounds refer to the those compounds which are capable, as such or in dissolved form, of rotating the plane of polarization of linear-polarized light passing through. Compounds having a stereogenic center are nonracemic mixtures of the two enantiomers, i.e. mixtures in which the two enantiomers are not present in equal parts. In the case of the conversion of compounds having more than one stereocenter, different diastereomers may be obtained which, each viewed alone, are to be regarded as optically active compounds.

Possible substituents bonded to asymmetrically substituted carbon atoms include: hydroxyl, alkoxy, amino, alkyl, aryl or chlorine substituents, and alkoxy substituents refers in particular to those whose organic radical bonded to the oxygen atom has from 1 to 8 carbon atoms, amino substituents may be present in the form of the free amine or preferably in protonated form as the ammonium salt and if appropriate having one or two organic radicals each having from 1 to 5 carbon atoms, the alkyl substituents have from 1 to 10 carbon atoms and the aryl substituents from 3 to 14 carbon atoms and may themselves bear substituents which are stable under the reaction conditions, and the aryl substituents may also have from 1 to 3 heteroatoms, for example N, S and/or O.

The substituents mentioned may in principle be attached at any possible point on the mono- or dicarboxylic acid to be converted. Preferred substrates in the context of the present invention are those which have at least one of the substituents mentioned which have on an asymmetric carbon atom in the α- or β-position, more preferably in the α-position to the acid function to be hydrogenated.

In the case of the conversion of dicarboxylic acids, the inventive hydrogenation reaction may, as desired, be conducted in such a way that either only one or both of the carboxylic acid functions or carboxylic acid derivative functions present in the substrate molecule are hydrogenated to the hydroxyl functions.

For example, the process according to the invention is suitable for converting optically active carboxylic acids or their acid derivatives of the formula I 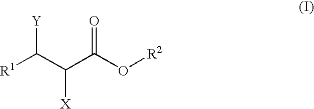
in which the radicals are each defined as follows:
R¹: straight-chain and branched C₁-C₁₂-alkyl, C₇-C₁₂-aralkyl or C₆-C₁₄-aryl, where the radicals mentioned may be substituted by NR³R⁴, OH, COOH and/or further groups stable under the reaction conditions,
R²: hydrogen, straight-chain or branched C₁-C₁₂-alkyl or C₃-C₈-cycloalkyl,
X, Y:
   each independently hydrogen, chlorine, NR⁵R⁶ or OR⁷, straight-chain or branched C₁-C₁₀-alkyl or C₆-C₁₄-aryl, with the proviso that at least one of the X or Y radicals is not hydrogen,
X and R¹ or Y and R¹:
   together may also be a 5 to 8-membered cycle,
R³, R⁴, R⁵ and R⁶:
   each independently hydrogen, straight-chain and branched C₁-C₁₂-alkyl, C₇-C₁₂-aralkyl, C₆-C₁₄-aryl, C₃-C₈-cycloalkyl or C₃-C₈-cycloalkyl in which one CH₂ group has been replaced by O or NR⁸,
R³ and R⁴, and R⁵ and R⁶:
   each independently together also —(CH₂)ₘ— where m is an integer from 4 to 7,
R¹ and R⁵:

together also —(CH₂)ₙ— where n is an integer from 2 to 6,
R⁷: hydrogen, straight-chain or branched C₁-C₁₂-alkyl or C₃-C₈-cycloalkyl,
R¹ and R⁷:
   together also —(CH₂)ₙ— where n is an integer from 2 to 6 and
R⁸: hydrogen, straight-chain or branched C₁-C₁₂-alkyl, C₇-C₁₂-aralkyl or C₆-C₁₄-aryl,
   or their acid anhydrides to the corresponding optically active alcohols.

The R¹ radicals may be varied widely and may also bear, for example, from 1 to 3 substituents stable under the reaction conditions such as NR³R⁴, OH and/or COOH.

Examples of R¹ radicals include the following:

C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl,

C₁-C₁₂-alkyl such as C₁-C₆-alkyl (mentioned above) or unbranched or branched heptyl, octyl, nonyl, decyl, undecyl or dodecadecyl,

C₇-C₁₂-aralkyl such as phenylmethyl, 1-phenylethyl, 2-phenylethyl, 1-phenylpropyl, 2-phenylpropyl or 3-phenylpropyl,

C₆-C₁₄-aryl such as phenyl, naphthyl or anthracenyl, where the aromatic radicals may be as substituents such as NR⁹R¹⁰, OH and/or COOH.

Examples of definitions for R² are as follows:

hydrogen, straight-chain or branched C₁-C₁₂-alkyl (as mentioned above) or C₃-C₈-cycloalkyl, for example cyclopropyl, cyclobutyl, cyclopentyl, cyclohexyl, cycloheptyl and cyclooctyl.

Instead of the carboxylic esters, the carboxylic acid derivatives used may also be the acid anhydrides.

The X and Y radicals are each independently chlorine, NR⁵R⁶ or OR⁷, where R⁵ and R⁶, just like R³ and R⁴, or R⁹ and R¹⁰, are each independently hydrogen, straight-chain and branched C₁-C₁₂-alkyl, in particular C₁-C₆-alkyl, C₇-C₁₂-aralkyl or C₆-C₁₄-aryl, in particular phenyl, or C₃-C₈-cycloalkyl (in each case as mentioned above for the R¹ and R² radicals), and where at least one of the X and Y radicals is not hydrogen.

The X and R¹ or Y and R¹ radicals may also together be a 5- to 8-membered, saturated or unsaturated and optionally substituted ring, for example a cyclopentyl, a cyclohexyl or a cyclooctyl radical.

The R³ and R⁴, R⁵ and R⁶, and R⁹ and R¹⁰ radicals may together each independently also be —(CH₂)ₘ— where m is an integer from 4 to 7, in particular 4 or 5. One CH₂ group may be replaced by O or NR⁸.

The R¹ and R⁵ radicals together may also be —(CH₂)ₙ— where n is an integer from 2 to 6.

The R⁷ radical is preferably hydrogen or straight-chain or branched C₁-C₁₂-alkyl or C₃-C₈-cycloalkyl, more preferably methyl, ethyl, 1-methylethyl, 1,1-dimethylethyl, hexyl, cyclohexyl or dodecyl. Together with R¹, it may also be —(CH₂)ₙ— where n is an integer from 2 to 6.

The process according to the invention is also suitable for converting optically active dicarboxylic acids or their acid derivatives, in particular those of the formula (II) 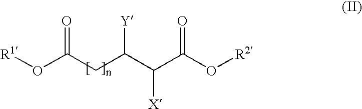
where
X′, Y′: each independently hydrogen, chlorine, NR^{5′}R^{6′} or OR^{7′}, straight-chain or branched C₁-C₁₀-alkyl or C₆-C₁₀-aryl, with the proviso that at least one of the X′ or Y′ radicals is not hydrogen,
R¹′, R²′: each independently hydrogen, straight-chain or branched C₁-C₁₂-alkyl or C₃-C₈-cycloalkyl and
n is an integer from 0 to 8
R⁵′, R⁶′: each independently hydrogen, straight-chain and branched C₁-C₁₂-alkyl, C₇-C₁₂-aralkyl, C₆-C₁₄-aryl, C₃-C₈-cycloalkyl or C₃-C₈-cycloalkyl, in which one CH₂ group is replaced by O or NR⁸′ and, together, is also —(CH₂)ₘ— where m is an integer from 4 to 7,
R^{7′}: hydrogen, straight-chain or branched C₁-C₁₂-alkyl or C₃-C₈-cycloalkyl and
R^{8′}: hydrogen, straight-chain or branched C₁-C₁₂-alkyl, C₇-C₁₂-aralkyl or C₆-C₁₄-aryl
   to the corresponding optically active hydroxy carboxylic acids or their acid derivatives or, in the case of the hydrogenation of both carboxylic acid functions, to the corresponding optically active substituted diols. For example, it is also possible to hydrogenate optically active hydroxy dicarboxylic acids to the corresponding optically active triols.

R¹′ and R²′ may, by way of example and each independently, assume the following definitions: hydrogen, straight-chain or branched C₁-C₁₂-alkyl (as specified above for radical R¹ in formula I) or C₃-C₈-cycloalkyl, for example, cyclopropyl, cyclobutyl, cyclopentyl, cyclohexyl, cycloheptyl and cyclooctyl.

Instead of the carboxylic esters, the carboxylic acid derivatives used may also be the acid anhydrides.

The X′ and Y′ radicals are each independently hydrogen, chlorine, NR^{5′}R^{6′} or OR^{7′}, where R^{5′} and R^{6′} are each independently hydrogen, straight-chain and branched C₁-C₁₂-alkyl, in particular C₁-C₆-alkyl, C₇-C₁₂-aralkyl or C₆-C₁₄-aryl, in particular phenyl, or C₃-C₈-cycloalkyl (in each case as specified above for the R¹ and R² radicals in formula I).

The R^{5′} and R^{6′} radicals may each independently together also be —(CH₂)ₘ where m is an integer from 4 to 7, in particular 4 or 5. One CH₂ group may be replaced by O or NR^{8′}.

The R^{7′} radical is preferably hydrogen or straight-chain or branched C₁-C₁₂-alkyl or C₃-C₈-cycloalkyl, more preferably methyl, ethyl, 1-methylethyl, 1,1-dimethylethyl, hexyl, cyclohexyl or dodecyl.

The optically active hydroxy carboxylic acids or diols obtainable by the process according to the invention by hydrogenating optically active dicarboxylic acids, for example those of the formula II, may, under suitable conditions, also form optically active cyclization products by intramolecular cyclization, for example lactones, lactams or cyclic ethers. Preferred cyclization products are the lactones and cyclic ethers, whose preparation in optically active form by hydrogenation of optically active dicarboxylic acids and subsequent cyclization also forms part of the subject matter of this invention. Preferred optically active lactones obtainable in the inventive manner starting from optically active dicarboxylic acids of the formula II are, for example, those of the formula III or IV 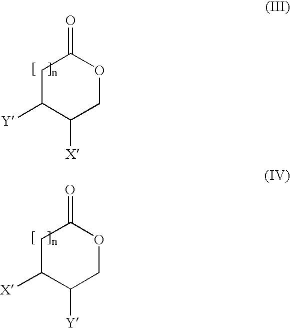
where the X′, Y′ radicals and n are each as defined for formula II.

Preferred cyclic ethers obtainable in optically active form in the inventive manner starting from optically active dicarboxylic acids of the formula II are, for example, those of the formula V or VI 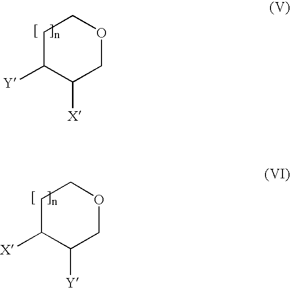
where the X′, Y′ radicals and n are each as defined for formula II.

In this way, the process according to the invention makes available, for example, the following lactones in optically active form: 2-hydroxy-γ-butyrolactone, 3-hydroxy-γ-butyrolactone, 2-chloro-γ-butyrolactone, 3-chloro-γ-butyrolactone, 2-amino-γ-butyrolactone, 3-amino-γ-butyrolactone, 2-methyl-γ-butyrolactone, 3-methyl-γ-butyrolactone, 3-hydroxy-δ-valerolactone, 4-hydroxy-δ-valerolactone.

Among these, particular preference in the context of the inventive preparative process is given to 3-hydroxy-γ-butyrolactone in optically active form.

Examples of cyclic ethers made available in optically active form by the process according to the invention include: 2-hydroxytetrahydrofuran, 2-methyltetrahydrofuran and 2-aminotetrahydrofuran.

Examples of preferred compounds obtainable in optically active form by the process according to the invention include:

1,2- and 1,3-amino alcohols, for example: α-alaninol, and also, in each case in the α- or β-form: leucinol, isoserinol, valinol, isoleucinol, serinol, threoninol, lysinol, phenylalaninol, tyrosinol, prolinol, and also the alcohols obtainable from the amino acids ornithine, citrulline, aspartine, aspartic acid, glutamine and glutamic acid, by converting the corresponding optically active α- or β-amino acids or their acid derivatives,

1,2- and 1,3-alkanediols, for example: 1,2-propanediol, 1,2-butanediol, 1,2-pentanediol, 1,3-pentanediol, by converting the corresponding optically active α- or β-hydroxy carboxylic acids or their acid derivatives,

1,2- and 1,3-chloroalcohols, for example 2-chloropropanol, by converting the corresponding optically active α- or β-chlorocarboxylic acids, α- or β-chlorodicarboxylic acids or their acid derivatives,

1,2- and 1,3-alkylalcohols, for example 2-methyl-1-butanol, 2,3-dimethylbutane-1,4-diol or 2-methylbutane-1,4-diol, by converting the corresponding optically active α- or β-alkylcarboxylic acids or their acid derivatives,

triols, for example 1,2,4-butanetriol, 1,2,5-pentanetriol, 1,2,6-hexanetriol, by converting the corresponding optically active α- or β-hydroxyhydroxydicarboxylic acids and

dihydroxycarboxylic acids or their acid derivatives, for example 3,4-dihydroxybutyric acid, by converting the corresponding optically active dicarboxylic acids.

Suitable for carrying out the inventive hydrogenation process are those catalysts whose active component comprises a noble metal selected from the group of the metals Pt, Pd, Rh, Ir, Ag, Au and at least one further element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu.

Preferred catalysts in the context of the process according to the invention are those whose active component comprises a noble metal selected from the group of the metals Pt, Pd, Rh, Ir, and at least one further element selected from the group of elements specified above. Among these further elements, preference is given to the elements Sn, Ge, Cr, Mo and W, particular preference to Sn.

Particularly preferred catalysts in the context of the process according to the invention comprise, in the active component, a noble metal selected from the group of the metals Pt, Pd, Rh, Ir, and at least one further element selected from the group of the elements Sn, Ge, Cr, Mo and W. Special preference is given to those catalysts whose active component comprises a noble metal selected from the group of the metals Pt, Pd, Rh, Ir, and, as the further component, Sn. Very particular preferred catalysts have an active component which comprises Pt and Sn.

The inventive catalysts may be used with good success as unsupported or as supported catalysts. Supported catalysts have the feature that the selected active component has been applied to the surface of a suitable support. To carry out the inventive hydrogenation process, particular preference is given to supported catalysts which have a high surface area and therefore require small amounts of the active metals.

The unsupported catalysts can be prepared, for example, by reducing a slurry and/or solution in aqueous or organic medium of the noble metal and of the further inventive active components in metallic form or in the form of compounds, for example oxides, oxide hydrates, carbonates, nitrates, carboxylates, sulfates, phosphates, halides, Werner complexes, organometallic complexes or chelate complexes or mixtures thereof.

When the catalysts are used in the form of supported catalysts, preference is given to supports such as charcoals, carbon blacks, graphites, high-surface activated graphites (HSAG), SiO₂, Al₂O₃, TiO₂, ZrO₂, SiC, clay earths, silicates, montmorillonites, zeolites or mixtures thereof. For use as support materials, particular preference is given to charcoals, graphites, HSAG, TiO₂ and ZrO₂.

In the case of the carbon-based supports (activated carbons, graphites, carbon blacks, HSAG), it is advantageous in accordance with the invention to treat the support material oxidatively with customary antioxidants, for example HNO₃, H₂O₂, O₂, air, O₃, ammonium persulfate, sodium hypochlorite, hypochlorous acid, perchloric acid, nitrate salts, and/or with acids such as HNO₃, H₃PO₄, HCl or HCOOH. Particular preference is given to pretreating with HNO₃, H₃PO₄ or HCOOH. The support may be treated before or during the application of the metals. The pretreatment allows activity and selectivity of the supported catalysts in the inventive hydrogenation to be improved.

The inventive supported catalysts typically comprises from about 0.01 to 30% by weight of a noble metal selected from the group of the metals Pt, Pd, Rh, Ir, Ag, Au in metallic form or in the form of compounds, and from 0.01 to 50% by weight, preferably from about 0.1 to 30% by weight and more preferably from about 0.5 to 15% by weight, of at least one further element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu in metallic form or in the form of a compound or mixtures thereof. The percentages by weight are in each case based on the total weight of the finished catalysts and calculated in metallic form.

The proportion of the noble metal selected from the group of the metals Pt, Pd, Rh, Ir, Ag, Au, calculated as the metal, is preferably from about 0.1 to 20% by weight, more preferably from about 0.5 to 15% by weight, based on the total weight of the finished supported catalyst.

The noble metal component used is typically an oxide, oxide hydrate, carbonate, nitrate, carboxylate, sulfate, phosphate or halide, preferably nitrate, carboxylate or halide.

The at least one further element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu, in addition to the noble metal selected from the group of the metals Pt, Pd, Rh, Ir, Ag, Au, is typically applied to the support material in the form of metal, oxides, oxide hydrates, carbonates, nitrates, carboxylates, sulfates, phosphates, Werner complexes, chelate complexes or halides. Preference is given to compounds of Sn, Ge, Cr, Mo or W, particular preference to Sn in the form of oxides or halides, for example SnCl₂, SnCl₄, SnO₂, GeCl₄ or GeO₂.

The application of the active components may be prepared in one or more steps by impregnation with an aqueous or alcoholic solution of the particular dissolved salts or oxides or of dissolved oxidic or metallic colloids, or by equilibrium adsorption in one or more steps of the salts or oxides dissolved in aqueous or alcoholic solution, or of dissolved oxidic or metallic colloids. Between individual equilibrium adsorption or impregnation steps, a drying step may in each case be carried out to remove the solvent and, if desired, a calcination step or reduction step.

The drying is advantageously carried out in each case at temperatures of from about 25 to about 350° C., preferably from about 40 to about 280° C., and more preferably from about 50 to about 150° C.

If desired, a calcination may be effected after each application or drying step at temperatures in the range from about 100 to 800° C., preferably from about 200 to about 600° C. and more preferably about 300 to about 500° C.

If desired, a reduction may be carried out after each application step.

In a particular embodiment of the preparation of the supported catalysts usable in accordance with the invention, an element selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu, in a first impregnation step, is applied to the support from the particular oxides, oxide hydrates, carbonates, nitrates, carboxylates, sulfates, phosphates, Werner complexes, chelate complexes or halides, then there is a drying step and, if desired, a calcination step and, if desired, a reduction step. Afterward, there is, if desired, a further impregnation with one or more elements selected from the group of the elements: Sn, Ge, Cr, Mo, W, Ti, Zr, V, Mn, Fe, Co, Ni, Cu, Zn, Ga, In, Pb, Bi, Ce, Pr, Nd, Pm, Sm, Eu, Gd, Tb, Dy, Ho, Er, Tm, Yb, Lu from the particular oxides, oxide hydrates, carbonates, nitrates, carboxylates, sulfates, phosphates, Werner complexes, chelate complexes or halides with subsequent drying and, if desired, calcination. In the last preparation step, the noble metal selected from the group of the metals Pt, Pd, Rh, Ir, Ag, Au is applied to the support in the form of nitrates, carboxylates or halides. Finally, there is a further drying step and, if desired, a calcination step.

A further means of preparing the inventive supported catalysts consists in the electroless deposition of a noble metal selected from the group of the metals Pt, Pd, Rh, Ir, Ag, Au and at least one further metallic component selected from the group of the elements: Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Tb, Dy, Ho, Er, Tm, Yb, Lu from the particular oxides, oxide hydrates, carbonates, nitrates, carboxylates, sulfates, phosphates, Werner complexes, chelate complexes or halides to the support material. The electroless deposition is advantageously effected in aqueous or alcoholic slurry of the support material and the particular metal compounds by adding reducing agents, for example alcohols or sodium hypophosphite, formic acid or alkali metal formates, in particular sodium formate. Particular preference is given to ethanol and NaH₂PO₂.

After the deposition, a drying step is advantageously carried out at temperatures in the range from about 25 to about 350° C., preferably from about 40 to about 280° C. and more preferably from about 50 to about 150° C.

If desired, a calcination may be effected after the deposition at temperatures in the range from about 100 to about 800° C., preferably from about 200 to about 600° C. and more preferably from about 300 to about 500° C.

The catalysts used in accordance with the invention are typically activated before used. In the case of the catalysts prepared by electroless deposition, this activation step may, if desired, be dispensed with. Preference is given to activating using hydrogen or a mixture of hydrogen and an inert gas, typically a mixture of H₂ and N₂. The activation is carried out at temperatures of from 100 to about 500° C., preferably from about 140 to about 400° C. and more preferably from about 180 to about 330° C. Activation is effected at pressures of from about 1 bar to about 300 bar, preferably from about 5 to about 200 bar and more preferably from about 10 to about 100 bar.

The catalysts usable in accordance with the invention typically have a specific surface area of from about 5 to 3000 m²/g, preferably from about 10 to about 1500 m²/g.

The inventive hydrogenation reaction typically proceeds in the presence of hydrogen at temperatures in the range from about 10 to about 300° C., preferably from about 30 to about 180° C. and more preferably from about 50 to 130° C. In general, a pressure of from about 1 to about 350 bar, preferably from about 10 to about 300 bar and more preferably from about 100 to about 300 bar is employed.

In the case of the inventive hydrogenation of optically active dicarboxylic acids to the corresponding optically active diols, preference is given to selecting a pressure of from about 150 to about 250 bar, more preferably from about 180 to about 250 bar and most preferably from about 200 to about 250 bar.

In a preferred embodiment of the process according to the invention, especially for hydrogenating amino-substituted substrates, the above-described optically active starting materials are hydrogenated in the presence of an organic or inorganic acid. In general, the addition of acid is from 0.5 to 1.5 equivalents, more preferably from 1 to 1.3 equivalents, based on 1 equivalent of any basic groups present in the starting materials. Useful organic acids include, for example, acetic acid, propionic acid and adipic acid. Preference is given to adding inorganic acids, especially sulfuric acid, hydrochloric acid and phosphoric acid. The acids may be used, for example, as such, in the form of aqueous solutions or in the form of their separately prepared salts with the starting materials to be hydrogenated, for example as sulfates, hydrogensulfates, hydrochlorides, phosphates, mono- or dihydrogenphosphates.

The optically active carboxylic acid or dicarboxylic acid to be converted may be used with good success in substance or in the form of an aqueous or organic solution. The hydrogenation may be carried out in suspension or in the liquid or gas phase in the fixed bed reactor in continuous mode.

In the case of a batchwise reaction, for example, from 0.1 to 50 g of the unsupported catalysts to be used in accordance with the invention or else from 0.1 to 50 g of supported catalysts to be used in accordance with the invention may be used based on 1 mole of optionally active starting compound used.

In a continuous process, the ratio of catalyst to starting compound to be converted is advantageously selected in such a way that a catalyst hourly space velocity in the range from about 0.005 to about 1 kg/I_{cat}h, preferably from about 0.02 to about 0.5 kg/I_{cat}h.

Suitable solvents for the reaction are, for example, the hydrogenation products themselves, water, alcohols, e.g. methanol, ethanol, propanol, butanol, ethers, e.g. THF or ethylene glycol ether. Preference is given to water or methanol or mixtures thereof as solvents.

The hydrogenation may be carried out in one or more stages in the gas or liquid phase. In the liquid phase, the suspension or fixed bed mode is possible. To carry out the process according to the invention, suitable reactors are all of those known by those skilled in the art to be suitable for carrying out hydrogenations, for example stirred tanks, fixed bed reactors, shaft reactors, tube bundle reactors, bubble columns or fluidized bed reactors.

The reaction is typically complete when no more hydrogen is taken up. Typically the reaction time is from about 1 to about 72 h.

The isolation and, if necessary, separation of the reaction products obtained may in principle be carried out by all customary processes known per se to those skilled in the art. Especially suitable for this purpose are extractive and distillative processes, and also the purification or isolation by crystallization.

The optically active reactants used or products obtained may be investigated for their enantiomeric purity by means of all methods known to those skilled in the art. Particularly suitable for this purpose are in particular chromatographic processes, especially gas chromatography processes or high-performance liquid chromatography (HPLC) processes. A suitable measure for determining the enantiomeric purity of the reactants or products is the enantiomeric excess (ee).

The process according to the invention features substantial suppression in the hydrogenation of the racemization of stereogenic centers of the substituted mono- or dicarboxylic acids used in optically active form as starting compounds. Accordingly, the enantiomeric excess of the products obtained in the process according to the invention typically corresponds substantially to the reactants used. Preference is given to selecting the reaction conditions in such a way that the enantiomeric excess of the desired product corresponds to at least 90%, more preferably to at least 95%, most preferably to at least 98%, of that of the starting compound used.

One advantage of the process according to the invention is that the known troublesome side reaction in those reactions, that of decarbonylation with release of carbon monoxide and its subsequent reduction to methane or other lower alkanes, is substantially suppressed. This leads to considerable safety advantages.

The following examples serve to illustrate the process according to the invention, but without restricting it in any way:

General procedure for the activation of the support materials by treating with an acid: 100 g of the selected support material are heated with 200 ml of the selected acid and 400 ml of water are heated to 100° C. with stirring for 45 min. After filtering off and washing with water, the activated support material is dried at 80° C. in a forced-air oven. When shaped bodies are used, the activation may also be carried out in a rotary evaporator or in a fixed bed reactor flowed through by the activation solution, in order to minimize the mechanical destruction of the support.

Catalyst 1 Preparation Method:

A 2 l stirred apparatus was initially charged with 25 g of Timrex® HSAG 100 (Timcal) pretreated with HCOOH, and 800 ml of ethanol, 1.7 g of Sn(CH₃COO)₂ and 3.4 g of Pt(NO₃)₂ in 800 ml of water, which are stirred at room temperature for 30 min. and then at 80° C. Subsequently, the mixture was filtered through a suction filter, washed and dried.

Catalyst 2 Preparation Method:

0.71 g of tungstophosphoric acid hydrate (H₃PW₁₂O₄₀xH₂O) and 4.6 g of Rh(NO₃)₃ were dissolved in water and made up to 18 ml of overall solution. This was used to impregnate 25 g of HCOOH activated Timrex® HSAG 100 in accordance with its water absorption. After drying for 16 hours, calcination was effected at 200° C. in a rotary tube.

Catalyst 3 Preparation Method:

1.6 g of GeCl₄ were dissolved in ethanol and made up to 23 ml of overall solution. This was used to impregnate 25 g of HCOOH-activated Timrex® HSAG 100 in accordance with its ethanol absorption. After drying for 16 hours, a second impregnation was effected, for which 4.6 g of Rh(NO₃)₃ were dissolved in water and made up to 18 ml of overall solution. This was used to impregnate the material for a second time in accordance with its water absorption, dried again for 16 hours and finally calcined at 400° C. in a rotary tube.

Catalyst 4 Preparation Method:

1 g of ammonium heptamolybdate (NH₄)₆Mo₇O₂₄xH₂O and 4.6 g of Rh(NO₃)₃ were dissolved in water and made up to 18 ml of overall solution. This was used to impregnate 25 g of HCOOH-activated Timrex® HSAG 100 in accordance with its water absorption. After drying for 16 hours, calcination is effected at 400° C. in a rotary tube.

### EXAMPLE 1

### Preparation of Optically Active Alaninol

An autoclave of capacity 300 ml was initially charged with 5 g of catalyst 1 together with 50 ml of water and stirred at 60 bar of hydrogen pressure and 270° C. for 2 hours. Subsequently, 24 g of L-alanine (>99% ee), 100 g of water and 13.2 g of H₂SO₄ were introduced and hydrogenation was effected at a pressure of from 180 to 200 bar and a temperature of 100° C. over a period of 12 h. After 12 h, the reaction effluent comprised 79.24 mol % of L-alaninol (ee>99.4) and 9 mol % of unconverted L-alanine.

### EXAMPLE 2

### Preparation of Optically Active β-hydroxy-γ-butyrolactone

An autoclave of capacity 300 ml was initially charged with 5 g of catalyst 1 together with 50 ml of water and stirred at 60 bar of hydrogen pressure and 270° C. for 2 hours. Subsequently, 24 g of malic acid and 120 g of water were introduced and hydrogenation was effected at a pressure of from 230 to 250 bar and a temperature of 100° C. over a period of 36 h. The reaction effluent comprised 22 mol % of 1,2,4-butanetriol (ee>98.2%), 57 mol % of β-hydroxy-γ-butyrolactone (ee>99%), 0.1 mol % of butanediol and 15 mol % of unconverted malic acid.

### EXAMPLE 3

### Preparation of Optically Active 1,2,4-butanetriol (BTO)

A batchwise autoclave (capacity 300 ml) was initially charged with 5 g of catalyst 2 with 50 ml of water, and stirred at hydrogen pressure 60 bar and 270° C. for 2 hours. Subsequently, 24 g of malic acid (MS) and 120 g of water were introduced and hydrogenation was effected at a pressure of from 230 to 250 bar and a temperature of 100° C. over a period of 36 h. The reaction effluent comprised 41 mol % of butanetriol, 9 mol % of hydroxybutyrolactone, 18 mol % of butanediol (BDO) and no unconverted malic acid.

### EXAMPLE 4

### Preparation of Optically Active 1,2,4-butanetriol (BTO)

A batchwise autoclave (capacity 300 ml) was initially charged with 5 g of catalyst 3 with 50 ml of water, and stirred at hydrogen pressure 60 bar and 270° C. for 2 hours. Subsequently, 24 g of malic acid (MS) and 120 g of water were introduced and hydrogenation was effected at a pressure of from 230 to 250 bar and a temperature of 100° C. over a period of 36 h. The reaction effluent comprised 38 mol % of butanetriol, (ee>98.6%), 6 mol % of hydroxybutyrolactone, 14 mol % of butanediol (BDO) and 6 mol % of unconverted malic acid.

### EXAMPLE 5

### Preparation of Optically Active 1,2,4-butanetriol (BTO)

A batchwise autoclave (capacity 300 ml) was initially charged with 5 g of catalyst 4 with 50 ml of water, and stirred at hydrogen pressure 60 bar and 270° C. for 2 hours. Subsequently, 24 g of malic acid (MS) and 120 g of water were introduced and hydrogenation was effected at a pressure of from 230 to 250 bar and a temperature of 100° C. over a period of 36 h. The reaction effluent comprised 59 mol % of butanetriol, (ee>98.6%), 17 mol % of butanediol (BDO) and no unconverted malic acid.

1. A process for preparing optically active hydroxy-, alkoxy-, amino-, alkyl-, aryl- or chlorine-substituted alcohols or hydroxy carboxylic acids having from 3 to 25 carbon atoms or their acid derivatives or cyclization products, the process comprising hydrogenating a substituted optically active mono- or dicarboxylic acids or their acid derivatives in the presence of a catalyst whose active component comprises a noble metal selected from the group of the metals Pt, Pd, Rh, Ir, Ag, Au and at least one further element selected from the group: consisting of Sn, Ge, Mo, W, Ti, Zr, V, Mn, Fe, Co, Ni, Cu, Zn, Ga, In, Pb, Bi, Cr, Ce, Pr, Nd, Pm, Sm, Eu, Gd, Th, Dy, Ho, Er, Tm, Yb, Lu.

2. The process according to claim 1, wherein the noble metal is selected from the group consisting of Pt, Pd, Rh and Ir.

3. The process according to claim 1, wherein the at least one further element is selected from the group consisting of: Sn, Ge, Cr, Mo and W.

4. The process according to claim 1, wherein the at least one further element is Sn.

5. The process according to claim 1, wherein the active component of the catalyst comprises Pt and Sn.

6. The process according to claim 1, wherein the substituted optically active mono- or dicarboxylic acids or their acid derivatives have at least one stereocenter in the α- or β-position to at least one carboxylic acid function or acid derivative function to be hydrogenated.

7. The process according to claim 1, wherein the hydrogenation results in a product selected from the group consisting of 1,2-propanediol, 1,2-butanediol, 1,2-pentanediol, 1,3-pentanediol, leucinol, isoserinol, valinol, isoleucinol, serinol, threoninol, lysinol, phenylalaninol, tyrosinol, prolinol, 2-chloropropanol, 2-methyl-1-butanol, 2,3-dimethylbutane-1,4-diol, 2-methylbutane-1,4-diol, 1,2,4-butanetriol, 1,2,5-pentanetriol, 1,2,6-hexanetriol, 2-hydroxy-γ-butyrolactone, 3-hydroxy-γ-butyrolactone, 2-chloro-γ-butyrolactone, 3-chloro-γ-butyrolactone, 2-amino-γ-butyrolactone, 3-amino-γ-butyrolactone, 2-methyl-γ-butyrolactone, 3-methyl-γ-butyrolactone, 3-hydroxy-\tilde{□}valerolactone, 4-hydroxy-□-valerolactone, 2-hydroxytetrahydrofuran, 2-methyltetrahydrofuran, 2-aminotetrahydrofuran 3,4-dihydroxybutyric acid.

8. The process according to claim 1, wherein the catalyst is a supported catalyst.

9. The process according to claim 8, wherein the catalyst, based on the total weight of the catalyst and calculated as the metal, comprises from 0.01 to 30% by weight of the noble metal and from 0.01 to 50% by weight of the at least one further element.

10. The process according to claim 8, wherein the support material is selected from ZrO₂, TiO₂, Al₂O₃, SiO₂, activated carbon, carbon blacks, graphites or high-surface area graphites.

11. The process according to claim 10, wherein the noble metal and the at least one further elements are applied to the support in the presence of a reducing agent.

12. The process according to claim 1, wherein the hydrogenation is conducted at a pressure of from 100 to 300 bar.

13. The process according to claim 12, wherein the hydrogenation is conducted at a temperature of from 30 to 180° C.

14. The process according to claim 1, wherein the hydrogenation is conducted in the presence of an acid.

15. The process according to claim 2, wherein at least one further element is selected from the group consisting of: Sn, Ge, Cr, Mo and W.

16. The process according to claim 2, wherein at least one further element is Sn.

17. The process according to claim 1, wherein the active component of the catalyst consists essentially of Pt and Sn.

18. A process for preparing optically active compounds, the process comprising hydrogenating a substituted optically active mono- or dicarboxylic acid or their corresponding acid derivatives in the presence of a catalyst whose active component comprises a noble metal selected from the group consisting of Pt, Pd, Rh and Ir, and at least one element selected from the group consisting of Sn, Ge, Cr, Mo and W.

19. The process according to claim 18, wherein the catalyst is a supported catalyst, the support material selected from ZrO₂, TiO₂, Al₂O₃, SiO₂, activated carbon, carbon blacks, graphites or high-surface area graphites.

20. The process according to claim 19, wherein the hydrogenation is conducted in the presence of a reducing agent, an acid or a reducing agent and an acid.

21. The process according to claim 18, wherein the active component of the catalyst consists essentially of Pt and Sn.
